(19) United States
(12) Patent Application Publication
(10) Pub. No.: US 2025/0067753 A1
(43) Pub. Date: Feb. 27, 2025

(54) Protein array-based in vitro transglutaminase assay platform for epitope mapping and immunogen design

(71) Applicant: Chen Liu, Pearland, TX (US)

(72) Inventor: Chen Liu, Pearland, TX (US)

(21) Appl. No.: 18/684,168

(22) PCT Filed: Aug. 19, 2022
(86) PCT No.: PCT/IB2022/000547

§ 371 Date: Feb. 15, 2024

(30) Foreign Application Priority Data
Aug. 20, 2021 (US) 63235155

(51) Int. Cl. G01N 33/68; C12Q 1/52; G16B 20/50

(52) U.S. Cl. CPC G01N 33/6878; C12Q 1/52; G16B 20/50

(57) ABSTRACT

The present invention provides methods for the determination and manipulation of immunodominant epitopes via respectively mapping and optimizing the glutamine-containing transglutaminase modification motifs. The invention discovers that the acyl donor glutamine-containing transglutaminase modification motifs of a peptide compose and correspond to the immunodominant epitope sequences recognized by the immunity, and the motifs' transglutaminase reactivity positively correlates with their immunogenicity. The invention further provides an approach for immunogen design wherein the immunogenicity of a protein could be manipulated through protein engineering measures changing the transglutaminase reactivity like amino acid substitution/mutation, deletion, or insertion, direct introduction/creation of glutamine residues/motifs, or any combination thereof.

### RELATED US PATENT APPLICATION

This application claims the benefits of U.S. Provisional Application No. 63/235,135 filed on Aug. 20, 2021.

### REFERENCES CITED

AbdAlla, S., H. Lother, A. Langer, Y. el Faramawy and U. Quitterer (2004). “Factor XIIIA transglutaminase crosslinks AT1 receptor dimers of monocytes at the onset of atherosclerosis.” Cell 119 (3): 343-354.
Achyuthan, K. E., A. Mary and C. S. Greenberg (1988). “The binding sites on fibrin (ogen) for guinea pig liver transglutaminase are similar to those of blood coagulation XIII. Characterization of the binding of liver transglutaminase to fibrin.” J Biol Chem 263 (28): 14296-14301.
Akimov, S. S., D. Krylov, L. F. Fleischman and A. M. Belkin (2000). “Tissue transglutaminase is an integrin-binding adhesion coreceptor for fibronectin.” J Cell Biol 148 (4): 825-838.
Andringa, G., K. Y. Lam, M. Chegary, X. Wang, T. N. Chase and M. C. Bennett (2004). “Tissue transglutaminase catalyzes the formation of alpha-synuclein crosslinks in Parkinson's disease.” Faseb j 18 (7): 932-934.
Bakker, E. N., A. Pistea and E. VanBavel (2008). “Transglutaminases in vascular biology: relevance for vascular remodeling and atherosclerosis.” J Vasc Res 45 (4): 271-278.
Balin, B. J., A. G. Loewy and D. M. Appelt (1999). “Analysis of transglutaminase-catalyzed isopeptide bonds in paired helical filaments and neurofibrillary tangles from Alzheimer's disease.” Methods Enzymol 309:172-186.
Bowness, J. M., J. E. Folk and R. Timpl (1987). “Identification of a substrate site for liver transglutaminase on the aminopropeptide of type III collagen.” J Biol Chem 262 (3): 1022-1024.
Bruce, S. E., I. Bjarnason and T. J. Peters (1985). “Human jejunal transglutaminase: demonstration of activity, enzyme kinetics and substrate specificity with special relation to gliadin and coeliac disease.” Clin Sci (Lond) 68 (5): 573-579.
Csosz, E., P. Bagossi, Z. Nagy, Z. Dosztanyi, I. Simon and L. Fesus (2008). “Substrate preference of transglutaminase 2 revealed by logistic regression analysis and intrinsic disorder examination.” J Mol Biol 383 (2): 390-402.
Csosz, E., B. Mesko and L. Fesus (2009). “Transdab wiki: the interactive transglutaminase substrate database on web 2.0 surface.” Amino Acids 36 (4): 615-617.
Davtyan, H., K. Zagorski, I. Petrushina, K. Kazarian, N. R. S. Goldberg, J. Petrosyan, M. Blurton-Jones, E. Masliah, D. H. Cribbs, M. G. Agadjanyan and A. Ghochikyan (2017). “MultiTEP platform-based DNA vaccines for alpha-synucleinopathies: preclinical evaluation of immunogenicity and therapeutic potency.” Neurobiol Aging 59:156-170.
Dieterich, W., T. Ehnis, M. Bauer, P. Donner, U. Volta, E. O. Riecken and D. Schuppan (1997). “Identification of tissue transglutaminase as the autoantigen of celiac disease.” Nat Med 3 (7): 797-801.
Dudek, S. M. and G. V. Johnson (1994). “Transglutaminase facilitates the formation of polymers of the beta-amyloid peptide.” Brain Res 651 (1-2): 129-133.
Eckert, R. L., M. T. Kaartinen, M. Nurminskaya, A. M. Belkin, G. Colak, G. V. Johnson and K. Mehta (2014). “Transglutaminase regulation of cell function.” Physiol Rev 94 (2): 383-417.
Gentile, V. and A. J. Cooper (2004). “Transglutaminases-possible drug targets in human diseases.” Curr Drug Targets CNS Neurol Disord 3 (2): 99-104.
Goldberg, A. L. and K. L. Rock (1992). “Proteolysis, proteasomes and antigen presentation.” Nature 357 (6377): 375-379.
Greenberg, C. S., P. J. Birckbichler and R. H. Rice (1991). “Transglutaminases: multifunctional cross-linking enzymes that stabilize tissues.” Faseb j 5 (15): 3071-3077.
Grosso, H. and M. M. Mouradian (2012). “Transglutaminase 2: biology, relevance to neurodegenerative diseases and therapeutic implications.” Pharmacol Ther 133 (3): 392-410.
Gundemir, S., G. Colak, J. Tucholski and G. V. Johnson (2012). “Transglutaminase 2: a molecular Swiss army knife.” Biochim Biophys Acta 1823 (2): 406-419.
Hitomi, K., M. Kitamura and Y. Sugimura (2009). “Preferred substrate sequences for transglutaminase 2: screening using a phage-displayed peptide library.” Amino Acids 36 (4): 619-624.
Houghten, R. A., C. Pinilla, S. E. Blondelle, J. R. Appel, C. T. Dooley and J. H. Cuervo (1991). “Generation and use of synthetic peptide combinatorial libraries for basic research and drug discovery.” Nature 354 (6348): 84-86.
Jeitner, T. M., N. A. Muma, K. P. Battaile and A. J. Cooper (2009). “Transglutaminase activation in neurodegenerative diseases.” Future Neurol 4 (4): 449-467.
Jeitner, T. M., J. T. Pinto and A. J. L. Cooper (2018). “Cystamine and cysteamine as inhibitors of transglutaminase activity in vivo.” Biosci Rep 38 (5).
Junn, E., R. D. Ronchetti, M. M. Quezado, S. Y. Kim and M. M. Mouradian (2003). “Tissue transglutaminase-induced aggregation of alpha-synuclein: Implications for Lewy body formation in Parkinson's disease and dementia with Lewy bodies.” Proc Natl Acad Sci USA 100 (4): 2047-2052.
Kárpáti, S., M. Sardy, K. Németh, B. Mayer, N. Smyth, M. Paulsson and H. Traupe (2018). “Transglutaminases in autoimmune and inherited skin diseases: The phenomena of epitope spreading and functional compensation.” Exp Dermatol 27 (8): 807-814.
Kahlem, P., H. Green and P. Djian (1998). “Transglutaminase action imitates Huntington's disease: selective polymerization of Huntingtin containing expanded polyglutamine.” Mol Cell 1 (4): 595-601.
Karpuj, M. V., H. Garren, H. Slunt, D. L. Price, J. Gusella, M. W. Becher and L. Steinman (1999). “Transglutaminase aggregates huntingtin into nonamyloidogenic polymers, and its enzymatic activity increases in Huntington's disease brain nuclei.” Proc Natl Acad Sci USA 96 (13): 7388-7393.
Keresztessy, Z., E. Csosz, J. Hársfalvi, K. Csomós, J. Gray, R. N. Lightowlers, J. H. Lakey, Z. Balajthy and L. Fésüs (2006). “Phage display selection of efficient glutamine-donor substrate peptides for transglutaminase 2.” Protein Sci 15 (11): 2466-2480.
Kim, S. Y., P. Grant, J. H. Lee, H. C. Pant and P. M. Steinert (1999). “Differential expression of multiple transglutaminases in human brain. Increased expression and cross-linking by transglutaminases 1 and 2 in Alzheimer's disease.” J Biol Chem 274 (43): 30715-30721.
Király, R., M. Demény and L. Fésus (2011). “Protein transamidation by transglutaminase 2 in cells: a disputed Ca2+-dependent action of a multifunctional protein.” Febs j 278 (24): 4717-4739.
Kiraly, R., M. Demeny and L. Fesus (2011). “Protein transamidation by transglutaminase 2 in cells: a disputed Ca2+-dependent action of a multifunctional protein.” Febs j 278 (24): 4717-4739.
Konno, T., T. Morii, A. Hirata, S. Sato, S. Oiki and K. Ikura (2005). “Covalent blocking of fibril formation and aggregation of intracellular amyloidgenic proteins by transglutaminase-catalyzed intramolecular cross-linking.” Biochemistry 44 (6): 2072-2079.
Laki, K., A. Benko and J. Farrell (1972). “Clot stabilization and atherosclerosis.” Ann N Y Acad Sci 202:235-239.
Lee, J. H., C. Song, D. H. Kim, I. H. Park, S. G. Lee, Y. S. Lee and B. G. Kim (2013). “Glutamine (Q)-peptide screening for transglutaminase reaction using mRNA display.” Biotechnol Bioeng 110 (2): 353-362.
Lesort, M., J. Tucholski, M. L. Miller and G. V. Johnson (2000). “Tissue transglutaminase: a possible role in neurodegenerative diseases.” Prog Neurobiol 61 (5): 439-463.
Liu, C., R. E. Kellems and Y. Xia (2017). “Inflammation, Autoimmunity, and Hypertension: The Essential Role of Tissue Transglutaminase.” Am J Hypertens 30 (8): 756-764.
Liu, C., R. Luo, S. E. Elliott, W. Wang, N. F. Parchim, T. Iriyama, P. S. Daugherty, S. C. Blackwell, B. M. Sibai, R. E. Kellems and Y. Xia (2015). “Elevated Transglutaminase Activity Triggers Angiotensin Receptor Activating Autoantibody Production and Pathophysiology of Preeclampsia.” J Am Heart Assoc 4 (12).
Liu, C., R. Luo, W. Wang, Z. Peng, G. V. W. Johnson, R. E. Kellems and Y. Xia (2019). “Tissue Transglutaminase-Mediated AT1 Receptor Sensitization Underlies Pro-inflammatory Cytokine LIGHT-Induced Hypertension.” Am J Hypertens 32 (5): 476-485.
Liu, C., W. Wang, N. Parchim, R. A. Irani, S. C. Blackwell, B. Sibai, J. Jin, R. E. Kellems and Y. Xia (2014). “Tissue transglutaminase contributes to the pathogenesis of preeclampsia and stabilizes placental angiotensin receptor type 1 by ubiquitination-preventing isopeptide modification.” Hypertension 63 (2): 353-361.
Liu, C. (2021).
“A protein microarray-based in vitro transglutaminase assay platform for epitope mapping and peptide discovery” BioRxiv 2021.05.12.443622
Lorand, L. and R. M. Graham (2003). “Transglutaminases: crosslinking enzymes with pleiotropic functions.” Nat Rev Mol Cell Biol 4 (2): 140-156. Lorand, L., T. Urayama, J. W. De Kiewiet and H. L. Nossel (1969). “Diagnostic and genetic studies on fibrin-stabilizing factor with a new assay based on amine incorporation.” J Clin Invest 48 (6): 1054-1064.
Malešević, M., A. Migge, T. C. Hertel and M. Pietzsch (2015). “A fluorescence-based array screen for transglutaminase substrates.” Chembiochem 16 (8): 1169-1174.
Masliah, E., E. Rockenstein, A. Adame, M. Alford, L. Crews, M. Hashimoto, P. Seubert, M. Lee, J. Goldstein, T. Chilcote, D. Games and D. Schenk (2005). “Effects of alpha-synuclein immunization in a mouse model of Parkinson's disease.” Neuron 46 (6): 857-868.
Michalek, M. T., E. P. Grant, C. Gramm, A. L. Goldberg and K. L. Rock (1993). “A role for the ubiquitin-dependent proteolytic pathway in MHC class I-restricted antigen presentation.” Nature 363 (6429): 552-554.
Molberg, O., S. N. McAdam, R. Korner, H. Quarsten, C. Kristiansen, L. Madsen, L. Fugger, H. Scott, O. Noren, P. Roepstorff, K. E. Lundin, H. Sjöström and L. M. Sollid (1998). “Tissue transglutaminase selectively modifies gliadin peptides that are recognized by gut-derived T cells in celiac disease.” Nat Med 4 (6): 713-717.
Muma, N. A. (2007). “Transglutaminase is linked to neurodegenerative diseases.” J Neuropathol Exp Neurol 66 (4): 258-263.
Nazif, T. and M. Bogyo (2001). “Global analysis of proteasomal substrate specificity using positional-scanning libraries of covalent inhibitors.” Proc Natl Acad Sci USA 98 (6): 2967-2972.
Nemes, Z., B. Devreese, P. M. Steinert, J. Van Beeumen and L. Fésüs (2004). “Cross-linking of ubiquitin, HSP27, parkin, and alpha-synuclein by gamma-glutamyl-epsilon-lysine bonds in Alzheimer's neurofibrillary tangles.” Faseb j 18 (10): 1135-1137.
Nemes, Z., G. Petrovski, M. Aerts, K. Sergeant, B. Devreese and L. Fésus (2009). “Transglutaminase-mediated intramolecular cross-linking of membrane-bound alpha-synuclein promotes amyloid formation in Lewy bodies.” J Biol Chem 284 (40): 27252-27264.
Norlund, M. A., J. M. Lee, G. M. Zainelli and N. A. Muma (1999). “Elevated transglutaminase-induced bonds in PHF tau in Alzheimer's disease.” Brain Res 851 (1-2): 154-163.
Nurminskaya, M. V. and A. M. Belkin (2012). “Cellular functions of tissue transglutaminase.” Int Rev Cell Mol Biol 294:1-97.
Parameswaran, K. N., P. T. Velasco, J. Wilson and L. Lorand (1990). “Labeling of epsilon-lysine crosslinking sites in proteins with peptide substrates of factor XIIIa and transglutaminase.” Proc Natl Acad Sci USA 87 (21): 8472-8475.
Pellois, J. P., X. Zhou, O. Srivannavit, T. Zhou, E. Gulari and X. Gao (2002). “Individually addressable parallel peptide synthesis on microchips.” Nat Biotechnol 20 (9): 922-926.
Rasmussen, L. K., E. S. Sørensen, T. E. Petersen, J. Gliemann and P. H. Jensen (1994). “Identification of glutamine and lysine residues in Alzheimer amyloid beta A4 peptide responsible for transglutaminase-catalysed homopolymerization and cross-linking to alpha 2M receptor.” FEBS Lett 338 (2): 161-166.
Sane, D. C., J. L. Kontos and C. S. Greenberg (2007). “Roles of transglutaminases in cardiac and vascular diseases.” Front Biosci 12:2530-2545.
Selkoe, D. J., C. Abraham and Y. Ihara (1982). “Brain transglutaminase: in vitro crosslinking of human neurofilament proteins into insoluble polymers.” Proc Natl Acad Sci USA 79 (19): 6070-6074.
Shainoff, J. R. and I. H. Page (1972). “Deposition of modified fibrinogen within the aortic intima.” Atherosclerosis 16 (3): 287-305.
Shinde, A. V. and N. G. Frangogiannis (2018). “Tissue transglutaminase in the pathogenesis of heart failure.” Cell Death Differ 25 (3): 453-456.
Siegel, M. and C. Khosla (2007). “Transglutaminase 2 inhibitors and their therapeutic role in disease states.” Pharmacol Ther 115 (2): 232-245.
Sollid, L. M. and B. Jabri (2011). “Celiac disease and transglutaminase 2: a model for posttranslational modification of antigens and HLA association in the pathogenesis of autoimmune disorders.” Curr Opin Immunol 23 (6): 732-738.
Sugimura, Y., M. Hosono, F. Wada, T. Yoshimura, M. Maki and K. Hitomi (2006). “Screening for the preferred substrate sequence of transglutaminase using a phage-displayed peptide library: identification of peptide substrates for TGASE 2 and Factor XIIIA.” J Biol Chem 281 (26): 17699-17706.
Suzuki, A. S., R. Yagi, M. Y. Kimura, C. Iwamura, K. Shinoda, A. Onodera, K. Hirahara, D. J. Tumes, R. Koyama-Nasu, S. E. lismaa, R. M. Graham, S. Motohashi and T. Nakayama (2020). “Essential Role for CD30-Transglutaminase 2 Axis in Memory Th1 and Th17 Cell Generation.” Front Immunol 11:1536.
Tagami, U., N. Shimba, M. Nakamura, K. Yokoyama, E. Suzuki and T. Hirokawa (2009). “Substrate specificity of microbial transglutaminase as revealed by three-dimensional docking simulation and mutagenesis.” Protein Eng Des Sel 22 (12): 747-752.
Tatsukawa, H., Y. Tani, R. Otsu, H. Nakagawa and K. Hitomi (2017). “Global identification and analysis of isozyme-specific possible substrates crosslinked by transglutaminases using substrate peptides in mouse liver fibrosis.” Sci Rep 7:45049.
Trombetta, E. S., M. Ebersold, W. Garrett, M. Pypaert and I. Mellman (2003). “Activation of lysosomal function during dendritic cell maturation.” Science 299 (5611): 1400-1403.
Turner, P. M. and L. Lorand (1989). “Complexation of fibronectin with tissue transglutaminase.” Biochemistry 28 (2): 628-635.
Wodzinska, J. M. (2005). “Transglutaminases as targets for pharmacological inhibition.” Mini Rev Med Chem 5 (3): 279-292.
Zhuang, R. and C. Khosla (2020). “Substrates, inhibitors, and probes of mammalian transglutaminase 2.” Anal Biochem 591:113560.

### BACKGROUND OF THE INVENTION

Transglutaminases (TGs) in mammalian cells are a family of crosslinking enzymes posttranslationally catalyzing the covalent isopeptide bond formation between the γ-carboxamide groups of a peptide-bound glutamine residue and a free amine or a peptide-bound lysine in a calcium dependent manner (Lorand and Graham 2003, Király, Demény et al. 2011, Gundemir, Colak et al. 2012, Eckert, Kaartinen et al. 2014). By catalyzing the formation of e-(γ-glutamyl)-lysine isopeptide bond in a calcium-dependent manner, transglutaminases function as a group of potent protein crosslinking enzymes in a myriad of important biological processes (Lorand and Graham 2003, Kiraly, Demeny et al. 2011, Gundemir, Colak et al. 2012, Eckert, Kaartinen et al. 2014). Functionally, TG-mediated isopeptide modification results in the stabilization and aggregation of substrate proteins by facilitating the assembly of supramolecular structure resistant to proteolysis (Greenberg, Birckbichler et al. 1991, Lorand and Graham 2003). Tissue transglutaminase (TG2), the most ubiquitous member of the enzyme family whose expression is regulated by inflammation and hypoxia (Liu, Kellems et al. 2017), serves as a unique sensor and effector for cellular calcium influx and energy depletion (Kiraly, Demeny et al. 2011, Nurminskaya and Belkin 2012). Activation of transglutaminase and TG-mediated isopeptide modification have been demonstrated to be pathogenically associated with severe human diseases including neurodegenerations (Lesort, Tucholski et al. 2000, Muma 2007, Jeitner, Muma et al. 2009, Grosso and Mouradian 2012), cardiovascular diseases (Sane, Kontos et al. 2007, Bakker, Pistea et al. 2008, Liu, Kellems et al. 2017, Shinde and Frangogiannis 2018), and autoimmune diseases (Sollid and Jabri 2011, Liu, Kellems et al. 2017, Kárpáti, Sárdy et al. 2018). Accumulation of pathogenic proteins crosslinked by transglutaminase has long been recognized as a hallmark of major neurodegenerative disorders including Alzheimer's disease (AD) (Selkoe, Abraham et al. 1982, Dudek and Johnson 1994, Rasmussen, Sørensen et al. 1994, Balin, Loewy et al. 1999, Kim, Grant et al. 1999, Norlund, Lee et al. 1999), Parkinson's disease (PD) (Junn, Ronchetti et al. 2003, Andringa, Lam et al. 2004), and Huntington's disease (HD) (Kahlem, Green et al. 1998, Karpuj, Garren et al. 1999). In cardiovascular diseases, transglutaminases contribute to the formation of atherosclerotic plaques by crosslinking extracellular matrix (ECM) proteins (Laki, Benkö et al. 1972, Shainoff and Page 1972, Bowness, Folk et al. 1987, Achyuthan, Mary et al. 1988, Turner and Lorand 1989, Akimov, Krylov et al. 2000) and to hypertensive disorders by modifying and sensitizing vasopressor receptors (AbdAlla, Lother et al. 2004, Liu, Luo et al. 2019). Transglutaminases are also well-known to participate in autoimmune responses through posttranslational modifications favoring the generation of neoantigens (Bruce, Bjarnason et al. 1985, Dieterich, Ehnis et al. 1997, Molberg, McAdam et al. 1998, Liu, Luo et al. 2015). Successful amelioration of key disease features by transglutaminase inhibitors in the pre-clinical animal models of related disorders not only establishes TG as an effective therapeutic target but also potentiates transglutaminase inhibitors as useful drugs for the disease treatment (Gentile and Cooper 2004, Wodzinska 2005, Siegel and Khosla 2007, Jeitner, Pinto et al. 2018, Zhuang and Khosla 2020). However, diagnosis of the related complications in patients usually accompanies a significant build-up of TG-crosslinked aggregates or plaques, and simply inhibiting the enzymes' activity is not sufficient to clean the build-ups. Given this concern, characterizing substrate-specific crosslinking sites and understanding the sequence preference of transglutaminase substrate would be of great importance in designing immunotherapeutic strategies capable of cleaning the aggregates to revoke the disease progression.

More importantly, previous studies indicate that TG2-mediated glutamine-lysine crosslinking represses the substrate's proteasome degradation through ubiquitination prevention (Liu, Wang et al. 2014). Since substrate ubiquitination is the prerequisite for proteasome and lysosome degradations, both of which participate in the generation of antigen peptides. TG2 crosslinking, which results in ubiquitination prevention (Liu, Wang et al. 2014) and proteolytic resistance (Lorand and Graham 2003), could be a crucial prerequisite for an antigen peptide's proteasomal (Goldberg and Rock 1992, Michalek, Grant et al. 1993) or lysosomal (Trombetta, Ebersold et al. 2003) survival in antigen processing. Aggregation of neurodegenerative proteins might be viewed as an extreme case where TG2 crosslinking saturates lysine residues for ubiquitination and helps the aggregates entirely escape the dual clearance by ubiquitin-proteasome system and autophagy, as well as immune recognition. In less saturated cases where ubiquitination and degradation still happen, TG2 crosslinking would save the modified parts of substrates from proteasome/lysosome digestion and aminopeptidase trimming, and thereby contribute to the genesis of immunogens. In this way, the transglutaminase modification motifs would compose the core parts of its immunodominant epitopes that usually refer to the small subset of the peptides preferred by both humoral and cell responses in the processing of a certain immunogen protein. The close match between TG2 modification motifs and immunodominant epitope sequences in alpha-synuclein presented in the current patent application implies a previously unrecognized essential role of TG2 in immunodominant determination. Consistently, endogenous TG2 is recently shown to be required for the generation of antigen-specific antibodies and memory Th cells (Suzuki, Yagi et al. 2020). Based on these findings, a universal, straightforward, and convenient approach for immunodominant epitope determination and optimization is established for next-generation immunotherapeutics.

To determine the enzymes' substrate preference and map the modification sites among various protein substrates, continued efforts using phage display (Keresztessy, Csosz et al. 2006, Sugimura, Hosono et al. 2006, Hitomi, Kitamura et al. 2009), mass spectrometry (Nemes, Devreese et al. 2004, Konno, Morii et al. 2005, Nemes, Petrovski et al. 2009, Tatsukawa, Tani et al. 2017), protein arrays (Lee, Song et al. 2013, Malešević, Migge et al. 2015), and bioinformatics tools (Csosz, Bagossi et al. 2008, Csosz, Meskó et al. 2009, Tagami, Shimba et al. 2009) have been witnessed. Although these studies greatly advanced our knowledge regarding the modification patterns of transglutaminases, a high-throughput platform with the efficiency of systems biology is still missing for the identification of substrate-specific TG modification sites. In this application, by combining the tagged amine donor dansyl-cadaverine-based in vitro TG assay (Lorand, Urayama et al. 1969) with a protein microarray (Pellois, Zhou et al. 2002) we established a platform for rapid and large-scale (up to 30000 reactions per chip) determination of the glutamine (Q)-containing TG modification motifs (Liu 2021). We tested the platform with peptides from neurodegenerative proteins including alpha-synuclein and superoxide dismutase 1 and mapped their primary modification sites for tissue transglutaminase (TG2). The Q109 in alpha-synuclein was characterized as the primary glutamine site for the enzyme modification. Other residues near the c-terminus including Q79 and Q134 were also identified as modification sites. Of particular interest, these modification motifs correspond to the epitope sequences found in animals with full-length protein immunization (Masliah, Rockenstein et al. 2005, Davtyan, Zagorski et al. 2017), in which antibodies recognize aa85-99, aa109-123, aa112-126, and aa126-138; B cells aa106-125; and T cells aa76-95 and aa106-125. The motifs with the primary modification site Q109 are the immunodominant epitopes present among all the antibody, B and T cell epitopes. Our data further indicate the glutamine and its follow-up five residues on its C terminal compose a minimal determinant motif for TG2 modification that could finally become core part of the substrate's epitope sequences in immunogen processing. To manipulate the TG2 modifications on a certain protein and screen for site-specific interfering peptides, we employed onchip amino-acid scanning (Houghten, Pinilla et al. 1991, Nazif and Bogyo 2001) and glutamine repeat addition methods for the optimization of modification motifs. By scanning the TG2 modification motif QQIV in the extracellular matrix protein fibronectin, we confirmed the platform's capability to serve TG-based peptide discovery and immunogen engineering.

### SUMMARY OF THE INVENTION

The invention provides methods for the determination and manipulation of immunodominant epitopes via respectively mapping and optimizing glutamine (Q)-containing transglutaminase modification motifs. With a protein microarray-based in vitro transglutaminase assay platform, the invention discovers that the acyl donor glutamine-containing transglutaminase modification motifs of a peptide substrate compose and correspond to the immunodominant epitope sequences recognized by the immunity in the peptide's antigen processing and the motifs' transglutaminase reactivity positively correlates with their immunogenicity. Based on this finding, the invention further provides an approach for de novo immunogen design wherein the immunogenicity of a protein of interest could be manipulated at will through protein engineering measures changing the peptide's transglutaminase activity including but not limited to amino acid substitution/mutation, deletion, or insertion, direct introduction/creation of glutamine (Q) residues/motifs, or any combination thereof.

In one aspect, the invention provides novel epitope mapping methods wherein the immunodominant epitope sequences of any protein or polypeptide of interest are determined via mapping the glutamine-containing transglutaminase modification motifs with classic in vitro transglutaminase assays using amine donor agents. In the present invention, the amine donor agent dansyl-cadaverine is covalently crosslinked in the assay to measure the transglutaminase reactivity of glutamine-containing motifs synthesized on the microchip. In some other embodiments, the amine donor agents could be lysine-containing peptides or their derivatives with chemical modifications including but not limited to Ac (acetyl)-Lys-Gly-MMAD (monomethyl auristatin D), Ac-Lys-Val-Cit-PABC (acetyl-lysine-valine-citrulline-p-aminobenzyloxycarbo ny)-MMAD, Ac-Lys-p-Ala-MMAD, Ac-Lys-Val-Cit-PABC-MMAE, Ac-Lys-Val-Cit-PABC-MMAF, and 5-FAM lysine; or amines or their derivatives with chemical modifications including but not limited to cystamine, spermidine, histamine, putrescine, dansyl-cadaverine, biotin cadaverine/5-(biotinamido) pentylamine, Alexa 488 cadaverine, 5-FITC cadaverine, Alexa 647 cadaverine, Alexa 350 cadaverine, 5-TAMRA cadaverine, 5-FAM cadaverine, SR101 cadaverine, amino-PEG3-C2-MMAD, amino-PEG6-C2-MMAD, amino-PEG3-C2-amino-non anoyl-MMAD, aminocaproyl-Val-Cit-PABC-MMAD, amino-PEG3-C2-Val-Cit-PA BC-MMAD, and amino-PEG6-C2-Val-Cit-PABC-MMAD. In addition to the protein microchip carrying synthesized glutamine-containing peptide substrates in the current application and other studies (Lee, Song et al. 2013, Malešević, Migge et al. 2015), the platforms for in vitro transglutaminase assay could also be phage display library (Keresztessy, Csosz et al. 2006, Sugimura, Hosono et al. 2006, Hitomi, Kitamura et al. 2009), peptide library, synthesized peptide strands, or cells/organisms expressing the target peptides. In some embodiments, the glutamine-containing transglutaminase modification motifs in the endogenous crosslinking, transamidation, or deamidation sites of tissues or cells could also be determined with structural biology approaches (e.g. X-ray crystallography, CryoEM) or mass spectrometry-related analytical methods for the epitope mapping. Based on findings in this application, in some embodiments bioinformatics tools or artificial intelligence algorithms predicting the glutamine-containing transglutaminase modification motifs (Csosz, Bagossi et al. 2008, Csosz, Meskó et al. 2009, Tagami, Shimba et al. 2009) could be employed for the immunodominant epitope determination.

In some embodiments, the transglutaminase-based epitope mapping approaches in this invention could be used to determine the crosslinking sites for the aggregates commonly seen in neurodegenerative and atherosclerotic plaques, and thereby create an immunogen blueprint for personalized and precise immunotherapy and vaccination clearing them. In some other embodiments, the transglutaminase-based approaches in this invention would directly identify the immunogen sequences presented by the host cells' MHC to the immunity from processed substrates of microbial pathogens, aging cells, or aberrant mutant proteins. In some other embodiments, the transglutaminase assay platform in this invention is able to characterize the optimal Q-containing tumor mutant peptide sequences with higher transglutaminase reactivity as the tumor-specific epitope candidates, since tumor mutant proteins would possess unique mutation-related peptide sequences favoring transglutaminase modifications that help them survive proteasome or lysosome cleavage as tumor neoantigens.

In another aspect, the invention further provides methods for de novo immunogen design wherein the immunogenicity of a protein of interest could be manipulated at will through protein engineering approaches changing the peptide's transglutaminase activity including but not limited to amino acid substitution/mutation, deletion, or insertion, direct introduction/creation of glutamine (Q) residues/motifs, or any combination thereof. In the current application, through scanning the residue directly following glutamine of the minimal determinant motif in the peptides QQIV and REQLYLDYNVFS, we obtained variants with significantly changed transglutaminase reactivity. We also introduced into the fibronectin peptide QQIV additional glutamine repeats with optimized adaptor sequence. With either of the approaches, we obtained variants with significantly elevated transglutaminase reactivity that are supposed to be associated with better immunogenicity. In some other embodiments, the glutamine-containing modification motifs could be truncated or elongated to enhance the enzyme reactivity of the original ones for better immunogenicity. In some embodiments the immunogenicity could be even created via adding glutamine residues or motifs to the peptides without glutamine residues.

In some embodiments, this invention could serve as a dynamic immunodominant epitope design tool overcoming the high mutation rate, genetic polymorphism, and low immunogenicity in therapeutic targets. For instance, mutations help viruses evade immune surveillance generated by the conventional vaccines, and compromised immunogenicity in conserved viral domains exacerbates the case. Following a global determination of immunodominant epitopes, improved and overlapping immunogens covering most mutational possibilities or less immunodominant but more conserved domains could be designed with the onchip positional scanning or glutamine introduction approach on the in vitro transglutaminase assay platform for next-generation universal vaccines with spatiotemporal coverage. In this way, a universal, straightforward, and convenient approach for immunodominant epitope determination and optimization could be established for next-generation immunotherapeutics treating most human diseases.

### DESCRIPTION OF THE DRAWINGS

FIG. 1. Peptide microarray-based in vitro TG2 assay platform for rapid and high-throughput identification of modification Q sites among proteins of interest. A. Assay flow chart for the Q mapping platform (Q: glutamine; CD: cadaverine; Dan: dansyl; Ab: antibody; TG2: tissue transglutaminase). B. Peptide layout strategy and naming rule on the microchip using the first glutamine (Q24) motif in human alpha-synuclein as the example. Each peptide where position of Q is defined as 0 and the one directly before or after it as −1 or 1 is named as protein name_Q residue # in the entire protein_N terminal residue # in the peptide˜C terminal residue # in the peptide.

FIG. 2. Identification of the glutamine-containing TG2 modification sites in SOD1 and alpha-synuclein with the high-throughput in vitro assay platform. A. Representative image of the protein microarray-based in vitro TG2 assay with the Q peptides in SOD1 showing that the first Q (Q16) peptides (red box) have a generally stronger reaction signal (red box) than the other two (Q23: yellow box; Q154: green box). Q16 residue in SOD1 (B) and Q109 in alpha-synuclein (C) are identified as the primary TG2 modification sites (p<0.05 vs other residues or mutant controls in B and C; n=triplicates; data=mean±SEM). The top 3 peptide hits of each Q residue in the TG2 reaction are plotted together with their Q to S mutants. D. Alignment of the top 10 hits of Q16 peptides in SOD1 and Q109 peptides in alpha-synuclein indicate their minimal determinant motifs.

FIG. 3. Onchip amino-acid scanning generates peptides with significantly changed reactivity with TG2. A. Pan-amino acid swapping at the I site of the peptide sequence QQIV generates 14 variants (red bars) with significantly higher TG2 reactivity and 1 lower (dark green) (*P<0.05 versus QQIV; n=triplicates; data=mean±SEM). B. Amino acid scanning at the L residue directly after the Q site of the TG2 substrate peptide REQLYLDYNVFS also obtained variants with significantly changed TG2 reactivity (*P<0.05 versus REQLYLDYNVFS; n=triplicates; data=mean±SEM).

FIG. 4. Addition of glutamine repeat elevates TG2 reactivity of the fibronectin peptide QQIV. A. Addition of glutamine repeat at the N terminal of the fibronectin peptide QQIV results in significant increase in dansyl-cadaverine incorporation as measured by fluorescent intensity on the chip (** P<0.01 versus QQIV; X=any of 20 amino acids; n=triplicates; data=mean±SEM). B. Majority (297 out of total 400) of the QQXXQQIV variants show a higher level of TG2 reactivity as measured by fluorescent intensity on the chip (the fluorescent intensity of QQIV is ˜5000 as indicated). C. Among the top 40 QQXXQQIV peptides with the highest fluorescent intensity, the peptides with a P at the third residue or I fourth appear most.

### EXAMPLES

### Methods

### Onchip In Vitro TG2 Assay

To identify the glutamine residues that can be modified by tissue transglutaminase on the peptide microchip, the synthesized peptide microchip was incubated with 100 μg/ml guinea pig liver tissue transglutaminase (Sigma) and 3 mM dansyl-cadaverine (Sigma) in 1 ml of TBS containing 5 mM Calcium Chloride and 1 mM DTT at 37 degree for 30 minutes. Afterwards, the peptide chip was washed at least 3 times with TBS. After washing off tissue transglutaminase and dansyl-cadaverine molecules bound on the synthesized peptides, the dansyl-cadaverine conjugated on the chip was tracked by rabbit anti-dansyl antibody (Invitrogen) followed by Alexa Fluor 594-labeled anti-rabbit secondary antibody (Invitrogen). Fluorescent microchip figures were quantified and analyzed with ArrayPro32. Original array figures and data are available upon request.

### Results

Mapping TG2 Modification Sites in Neurodegenerative Proteins with High-Throughput In Vitro Assay Platform

To establish a high-throughput assay platform for the rapid and large-scale identification of TG2 modification sites in disease-related proteins, we synthesized on microchips the glutamine-bearing motifs in superoxide dismutase 1 (SOD1) and alpha-synuclein, the pathogenic proteins in amyotrophic lateral sclerosis (ALS) and Parkinson's disease, respectively.

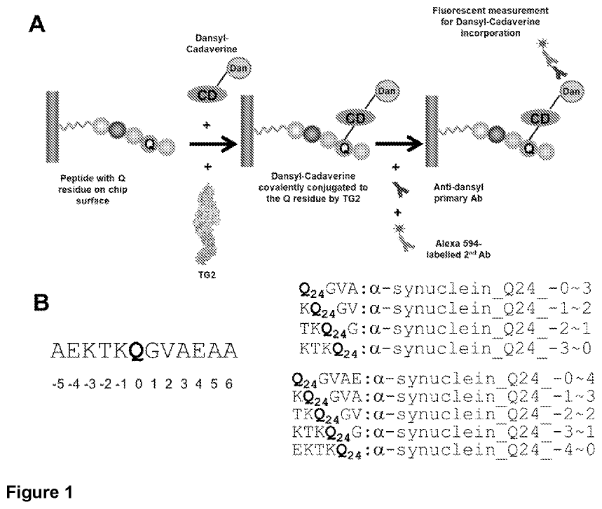

To characterize the glutamine-bearing TG2 modification motifs on these synthesized peptides, dansyl-cadaverine, a well-established amine donor in transglutaminase reaction, was covalently conjugated to the glutamine residues on the peptide chip by purified TG2 with the help of calcium (FIG. 1A). After washing off tissue transglutaminase and dansyl-cadaverine molecules bound on the synthesized peptides, the dansyl-cadaverine conjugated on the chip was probed by anti-dansyl antibody followed by Alexa Fluor 594-labeled anti-rabbit secondary antibody. In this way the level of dansyl-cadaverine incorporation on a certain peptide was measured by the fluorescent intensity.

Each peptide synthesized on the chip has a length of at least 4 amino acids, and its maximum length could be up to 12 mer. And the glutamine residue needs to appear in each position of the peptide once to ensure the thorough coverage of the screening (FIG. 1B). In this way the peptide screening may also pattern the substrate sequence optimal for the modification. For example, in human alpha-synuclein the surrounding sequence of the first glutamine is AEKTKQ₂₄GVAEAA (FIG. 1B). So the sequences for its 4 mer peptides would be QGVA, KQGV, TKQG, and KTKQ, and those for 5 mer would be QGVAE, KQGVA, TKQGV, KTKQG, and EKTKQ. Up to 11 residues on either N or C side of the glutamine is covered on the chip. Each peptide is named as protein name_Q residue # in the protein_N terminal residue # in the peptide˜C terminal residue # in the peptide (In the peptide position of Q is defined as 0, and the one directly before or after it as −1 or 1). So the 4 mer peptide KQ₂₄GV is named as α-synuclein_Q24_-1˜2 (FIG. 1B). Therefore, for each glutamine residue in a given protein, the initial number of peptide variants synthesized on the chip will be 4+5+6+7+8+9+10+11+12=72. The corresponding peptides with Q to S swap are also synthesized on the same chip as negative controls.

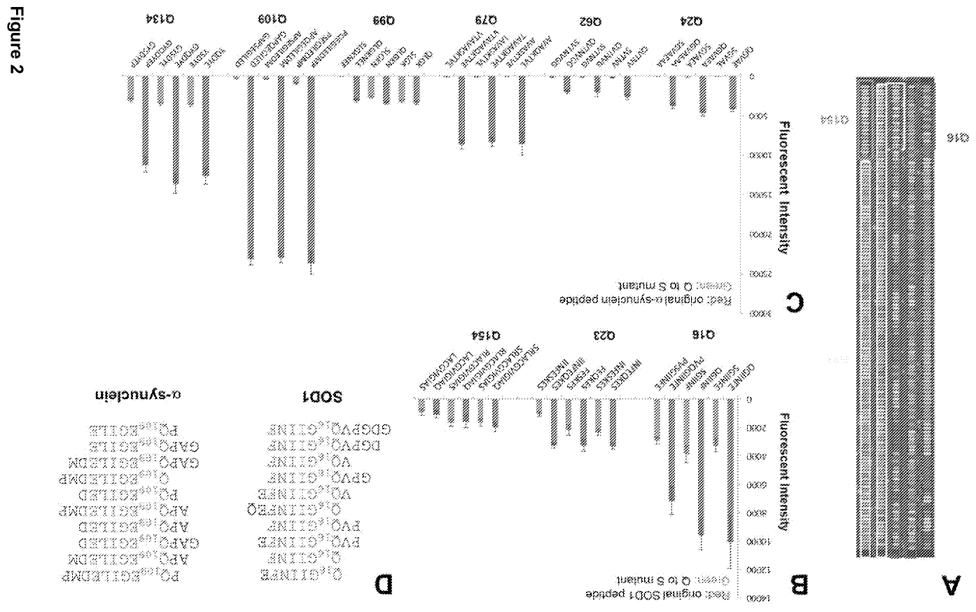

With this approach we identified Q16 in superoxide dismutase 1 and Q109 in alpha-synuclein as the primary modification sites for TG2 (FIGS. 2B and 2C). In human SOD1 protein there are 3 glutamine residues including Q16, Q23, and Q154. Compared with Q23 and Q154 counterparts, the Q16 peptides with the 5 mer motif directly following the glutamine residue (QGIINF) showed significantly higher fluorescent intensities (>6000), and the Q to S swap could effectively reduce their fluorescent levels, indicating the Q16 residue is the most probable TG2 modification site in the protein (FIG. 2B). Similarly, among the 6 glutamine residues in human alpha-synuclein protein, the Q109 residue with its follow-up 5 mer motif (QEGILE) elicited the strongest dansyl-cadaverine incorporation signal and thereby was identified as the TG2 modification site of the protein (FIG. 2C). Aligned data with top hits from SOD1 and alpha-synuclein collectively suggest that the Q and its follow-up 5 residues compose a minimal determinant motif for TG2 modification (FIG. 2D), which is further confirmed by the peptides with truncated minimal determinant motifs (not shown).

Optimizing TG2 Modification Sites with the Onchip Amino-Acid Scanning

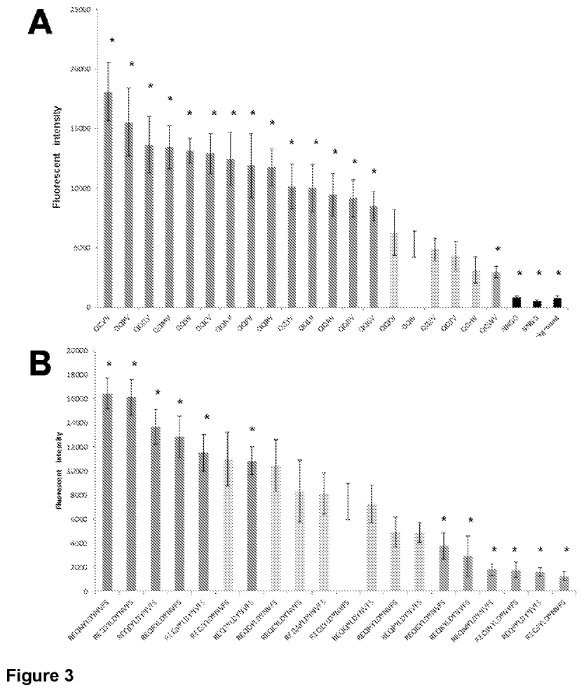

The small peptide QQIV is a transglutaminase substrate identified in the extracellular matrix protein fibronectin. As an amine acceptor, the peptide has been demonstrated to be an effective competitive inhibitor for the transglutaminase reaction (Parameswaran, Velasco et al. 1990). Given this, we chose it as one of the positive substrate peptides on the peptide chip. On our assayed chip the QQIV peptide showed a fluorescent intensity of ˜5000 which is much higher than those (˜800) of four-residue negative control peptides without glutamine residues and the chip background (FIG. 3A). These results confirmed the efficiency and specificity of the dansyl-cadaverine incorporation in our assay system. To scan for a more preferred TG2 substrate and thereby for a more optimized competitive inhibitor than QQIV, we swapped the isoleucine (I) residue in this small peptide to every other amino acid. Except one variant with reduced transglutaminase reactivity, 14 out of the 19 swapped peptides show significantly higher fluorescent signals and the improvement could be up to 3 folds (FIG. 3A), suggesting their better candidacy for competitive inhibitor and substrate of TG2 modification than the original QQIV. However, transglutaminase reactivity of most of these 14 mutants was significantly compromised when the hydrophobic V residue was changed to the hydrophilic G (data not shown). To further test the amino acid-scanning approach with the minimal determinant motif for TG2 modification, we swapped to any other amino acid the first leucine residue in the 12-mer small peptide sequence REQLYLDYNVFS, a known TG2 substrate found in a phage display library. Through pan-amino acid scanning at the L residue we found 6 variants (L to N, S, E, R, V or T) with significantly higher transglutaminase reactivity and 6 (L to G, K, M, W, Y or F) with lower reactivity (FIG. 3B). Taken together, our result indicates that the residues within the minimal determinant motif could be reasonable targets for the optimization of modification site and the design of substrate-specific interfering peptides.

Elevating Peptide's Reactivity with Transglutaminase by Adding Glutamine Repeats

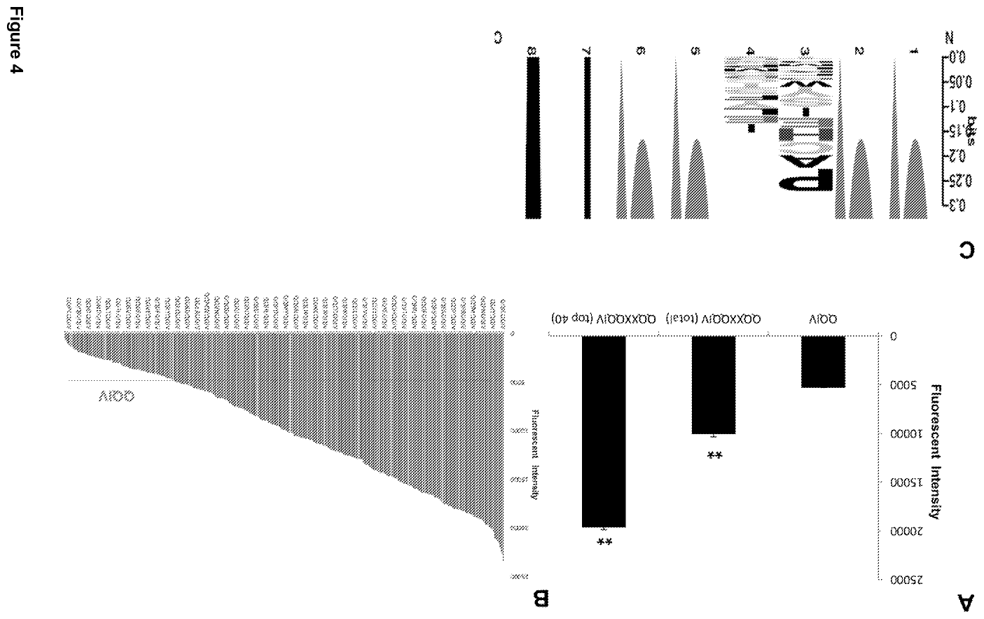

Characterized in neurodegenerative complications like the polyglutamine diseases, glutamine repeats elicit excellent TG substrate properties in polypeptides by functioning as efficient amine acceptors, and thereby are considered as a biochemical cause for the pathogenesis. To test this feature on our platform, we randomly added the double glutamine repeat motif QQXX (X stands for any of the 20 amino acids) at the N terminal of the peptide QQIV. Consistent with previous findings, the addition of double glutamine repeat motif resulted in a significant upregulation of the TG2-mediated conjugation of dansyl-cadaverine as the average fluorescent intensity (˜10000) of the 400 QQXXQQIV peptides is two folds higher than that (˜5000) of the original QQIV peptide (FIG. 4A) and 297 out of the 400 QQXXQQIV peptides show a fluorescent intensity of more than 5000 on our assayed chip (FIG. 4B). With an average fluorescent intensity of ˜20000 (FIG. 4A), the top 40 QQXXQQIV peptides could serve as competitive inhibitors to block TG2's modification on the QQIV motif of fibronectin. And among them, the peptides with P at the third residue or I at the fourth show up at most times (FIG. 4C), indicating a preferred pattern for the linker region between glutamine repeats.

### Summary and Conclusions

In this application we present a protein microarray-based in vitro TG assay platform for fast and high throughput identification of the glutamine (Q)-containing TG modification motifs. We tested the platform with peptides from neurodegenerative proteins including alpha-synuclein and superoxide dismutase 1 and mapped their primary modification sites for tissue transglutaminase (TG2). The Q109 in alpha-synuclein was characterized as the primary glutamine site for the enzyme modification. Other residues near the c-terminus including Q79 and Q134 were also identified as modification sites. Of particular interest, these modification motifs correspond to the epitope sequences found in animals with full-length protein immunization (Masliah, Rockenstein et al. 2005, Davtyan, Zagorski et al. 2017), in which antibodies recognize aa85-99, aa109-123, aa112-126, and aa126-138; B cells aa106-125; and T cells aa76-95 and aa106-125. The motifs with the primary modification site Q109 are the immunodominant epitopes present among all the antibody, B and T cell epitopes. Our data further indicate the glutamine and its follow-up five residues on its C terminal compose a minimal determinant motif for TG2 modification that could finally become core part of the substrate's epitope sequences in immunogen processing. To manipulate the transglutaminase reactivity on a certain peptide for a desired immunogenicity, we employed onchip amino-acid scanning (Houghten, Pinilla et al. 1991, Nazif and Bogyo 2001) and glutamine repeat addition methods for the optimization of modification motifs. By scanning the TG2 modification motif QQIV in the extracellular matrix protein fibronectin, we confirmed the platform's capability to serve TG-based peptide discovery and immunogen engineering.

What is claimed is:

1. Use of mapping the acyl donor glutamine-containing transglutaminase modification motifs with amine donor agent-based transglutaminase assays as an approach for the determination of immunodominant epitopes or epitope mapping. Wherein the platform for the glutamine-containing peptides could be peptide microarray/library, phage display library, synthesized peptide strands, or cells/organisms expressing the target peptides. Wherein the amine donor agents could be lysine-containing peptides or their derivatives with chemical modifications including but not limited to Ac (acetyl)-Lys-Gly-MMAD (monomethyl auristatin D), Ac-Lys-Val-Cit-PABC (acetyl-lysine-valine-citrulline-p-aminobenzyloxyca rbony)-MMAD, Ac-Lys-p-Ala-MMAD, Ac-Lys-Val-Cit-PABC-MMAE, Ac-Lys-V al-Cit-PABC-MMAF, and 5-FAM lysine; or amines or their derivatives with chemical modifications including but not limited to cystamine, spermidine, histamine, putrescine, dansyl-cadaverine, biotin cadaverine/5-(biotinamido) pentylamine, Alexa 488 cadaverine, 5-FITC cadaverine, Alexa 647 cadaverine, Alexa 350 cadaverine, 5-TAMRA cadaverine, 5-FAM cadaverine, SR 101 cadaverine, amino-PEG3-C2-MMAD, amino-PEG6-C2-MMAD, amino-PEG3-C2-amino-nonanoyl-MMAD, aminocaproyl-Val-Cit-PABC-MMAD, amino-PEG3-C2-Val-Cit-PABC-MMAD, and amino-PEG6-C2-Val-Cit-PABC-MMAD.

2. Use of protein engineering approaches changing the acyl donor glutamine-containing peptide's transglutaminase reactivity in amine donor agent-based transglutaminase assays as an approach to manipulate the immunogenicity of immunotherapeutic target peptides. Wherein the protein engineering approaches include but are not limited to amino acid substitution/mutation, deletion, or insertion, direct introduction/creation of glutamine (Q) residues/motifs, or any combination thereof.

3. Use of bioinformatics tools, artificial intelligence algorithms and/or molecular dynamics programs predicting and/or simulating the acyl donor glutamine-containing transglutaminase modification motifs as an approach to determine and manipulate the immunogenicity of immunotherapeutic target peptides.

4. Use of structural biology approaches (e.g. X-ray crystallography, CryoEM) or mass spectrometry-related analytical methods determining acyl donor glutamine-containing transglutaminase modification motifs in the endogenous crosslinking, transamidation, or deamidation sites of protein samples, tissues or cells.

5. Use of mutant acyl donor glutamine-containing transglutaminase modification motifs whose reactivity is significantly elevated in the approaches of claim 1, 3, or 4 as the differential immunodominant epitopes or immunogens for the immunotherapeutics targeting the cells carrying mutations like cancer cells.

6. Use of overlapping acyl donor glutamine-containing transglutaminase modification motifs detailed in FIG. 1B of this application as the template for the design of overlapping immunogen covering the same glutamine domain.

7. Use of alpha-synulcein and superoxide dismutase 1-derived synthetic peptides whose fluorescent intensity in the dansyl-cadaverine-based in vitro transglutaminase assay of this application is above 5000 artificial unit as the immunogen/antigens in immunotherapies and vaccines. Wherein the sequences of synthetic peptides include QEGI, QEGIL, QEGILE, QEGILED, QEGILEDM, QEGILEDMP, QEGILEDMPV, QEGILEDMPVD, QEGILEDMPVDP, PQEGI, PQEGIL, PQEGILE, PQEGILED, PQEGILEDM, PQEGILEDMP, PQEGILEDMPV, PQEGILEDMPVD, APQEGI, APQEGIL, APQEGILE, APQEGILED, APQEGILEDM, APQEGILEDMP, APQEGILEDMPV, GAPQEGI, GAPQEGIL, GAPQEGILE, GAPQEGILED, GAPQEGILEDM, GAPQEGILEDMP, EGAPQEGI, EGAPQEGIL, EGAPQEGILE, EGAPQEGILED, EGAPQEGILEDM, EEGAPQEGI, EEGAPQEGIL, EEGAPQEGILE, EEGAPQEGILED, NEEGAPQEGI, NEEGAPQEGIL, NEEGAPQEGILE, KNEEGAPQEGIL, QDYE, QDYEP, QDYEPE, QDYEPEA, YQDY, YQDYE, YQDYEP, YQDYEPE, YQDYEPEA, GYQDY, GYQDYE, GYQDYEP, GYQDYEPE, GYQDYEPEA, EGYQ, EGYQD, EGYQDY, EGYQDYE, EGYQDYEP, EGYQDYEPE, EGYQDYEPEA, EEGYQ, EEGYQD, EEGYQDY, EEGYQDYE, EEGYQDYEP, EEGYQDYEPE, EEGYQDYEPEA, SEEGYQ, SEEGYQD, SEEGYQDY, SEEGYQDYE, SEEGYQDYEP, SEEGYQDYEPE, SEEGYQDYEPEA, PSEEGYQ, PSEEGYQD, PSEEGYQDY, PSEEGYQDYE, PSEEGYQDYEP, PSEEGYQDYEPE, MPSEEGYQ, MPSEEGYQD, MPSEEGYQDY, MPSEEGYQDYE, MPSEEGYQDYEP, EMPSEEGYQ, EMPSEEGYQD, EMPSEEGYQDY, EMPSEEGYQDYE, YEMPSEEGYQ, YEMPSEEGYQD, YEMPSEEGYQDY, QKTVE, VAQKTVE, AVAQKTVE, AVAQKTVEG, TAVAQKTVE, TAVAQKTVEG, VTAVAQKTVE, VTAVAQKTVEG, GVTAVAQKTVE, GVTAVAQKTVEG, TGVTAVAQKTVE, QGIINFE, QGIINF, PVQGIINFE, PVQGIINF, QGIINFEQ, VQGIINFE, GPVQGIINF, VQGIINF, DGPVQGIINF, GDGPVQGIINF, and QGII.
